(19) United States
(12) Patent Application Publication
(10) Pub. No.: US 2018/0100841 A1
(43) Pub. Date: Apr. 12, 2018

(54) Micro Sensor Package and Manufacturing Method Thereof

(71) Applicant: Point Engineering Co., Ltd., Asan-si (KR)

(72) Inventors: Bum Mo Ahn, Suwon-si (KR); Seung Ho Park, Hwaseong-si (KR); Sung Hyun Byun, Hwaseong-si (KR)

(21) Appl. No.: 15/718,555

(22) Filed: Sep. 28, 2017

(30) Foreign Application Priority Data
Oct. 10, 2016 (KR) 10-2016-0130541

(51) Int. Cl. G01N 33/00; G01N 27/14

(52) U.S. Cl. CPC G01N 33/0027; G01N 27/14

(57) ABSTRACT

There is provided a micro sensor package, wherein a sensor platform and a cover are side-to-side surface-bonded through an adhesive layer so as to prevent unfiltered gas from being introduced to the sensor electrode.

### CROSS-REFERENCE TO RELATED PATENT APPLICATION

This U.S. non-provisional patent application claims priority under 35 U.S.C. § 119 of Korean Patent Application No. 10-2016-0130541 filed on Oct. 10, 2016 in the Korean Patent Office, the entire contents of which are hereby incorporated by reference.

### BACKGROUND

### 1. Technical Field

The present invention relates to a micro sensor package and a manufacturing method thereof, more particularly, relates to a micro sensor package and a manufacturing method thereof wherein a sensor platform and a cover are side-to-side surface-bonded through an adhesive layer.

### 2. Description of Related Art

A conventional miniature package for a gas sensor capable of sensing the amount of gas is shown in FIG. 1, which will be briefly described as follows.

A chip mounting portion 2 having a predetermined depth is formed at a central portion of a rectangular frame 1 made of an insulating material, and a sensor chip 4 is attached to the bottom surface of the chip mounting portion 2 with an epoxy 3.

A plurality of circuit lines 5 is formed inside of the frame 1, and a step portion 6 having a predetermined height along the inner circumferential surface is formed at the inner side edge of the chip mounting portion 2.

An inner terminal 5a extending from the one end of the circuit line 5 is formed on the step portion 6, and an outer terminal 5b extending from the other end (of the circuit line 5) is formed on the bottom edge of the frame 1.

A sensing film 16 for sensing gas is formed at the center portion of the upper surface of the sensor chip 4, and a plurality of sensor terminals 11 is formed at the edges for transmitting the resistance change detected by the sensing film 16 to the outside, and the sensor terminal 11 and the inner terminal 5a of the circuit line 5 are electrically connected by a silver paste 12, respectively.

A cap 13 is attached to the upper side of the frame 1 with an adhesive 14 so that the chip mounting portion 2 is covered, and in the cap 13, coupled in such a way, a plurality of gas holes 15 is formed so that gas can be introduced into the chip mounting portion 2.

In the micro-sized package for a gas sensor configured as described above, when a gas is introduced to inside the chip mounting portion 2 through the gas holes 15 in the cap 13, the resistance value of the sensing film 16 formed on the upper surface of the sensor chip 4 is varied due to the introduced gas, and the changing resistance values are transferred to a control unit (not shown) via the circuit lines 5, thereby measuring the amount of the gas.

Such a gas sensor is also provided with a heater, but the sensor chip 4 has a high thermal conductivity, so that there is a problem that a high power is required when the temperature needs to be raised to a high temperature.

In addition, since the lower surface of the cap 13 and the upper surface of the frame 1 are bonded through the adhesive 14, there is a problem that the measurement accuracy is decreased because unfiltered gas is introduced through between the cap 13 and the frame 1.

### SUMMARY

1. Technical Problem

An objective of the present invention devised for solving the above described problems, is to provide a micro sensor package and a manufacturing method thereof for preventing unfiltered gas from being introduced into the package.

2. Solution to Problem

To achieve above described objective, a micro sensor package of the present invention is characterized in that and comprises: a sensor platform; a sensing chip comprising a sensor electrode formed in the sensor platform; a cover covering the sensor electrode; and an adhesive layer formed on the side surfaces of the sensor platform and the cover, for bonding the sensor platform and the cover.

A substrate electrically connected to the sensing chip may further be included.

The adhesive layer is also formed on the upper surface of the substrate so that the sensing chip and the substrate can be bonded together.

In the sensor platform, a plurality of first pores may be formed along the up-down direction.

In the cover, a plurality of second pores for supplying a gas to the sensing chip may be formed along the up-down direction.

A cavity disposed with at least a portion of the sensor electrode is formed in the cover, and the cavity is penetratingly formed along the up-down direction, and a filter that covers the cavity may further be included.

A plurality of fourth pores is penetratingly formed in the filter along the up-down direction and the fourth pores may communicate with the cavity.

The adhesive layer is also formed on the side surface of the filter so that the filter can be bonded with the cover.

In the sensor platform, a heater electrode is formed, and the heater electrode comprises a heating wire disposed closer to a sensor wiring than a sensor electrode pad of the sensor electrode, and an air gap for surrounding the heating wire is formed in the sensor platform, and a porous layer disposed below the sensing chip and is penetratingly formed with a plurality of third pores along the up-down direction may further be included.

In the sensor platform, a heater electrode is formed, and the heater electrode comprises a heating wire disposed closer to a sensor wiring than a sensor electrode pad of the sensor electrode, and an air gap that surrounds the heating wire is formed in the sensor platform, and the upper side of the air gap is open, and a plurality of first pores may be disposed below the air gap.

The first pores are penetratingly formed along the up-down direction, and a first connecting portion which is electrically connected to the sensor electrode may be formed inside at least a portion of the plurality of first pores.

To achieve above described objective, a micro sensor package of the present invention is characterized in that and comprises: a sensor platform; a sensor electrode disposed above the sensor platform; a cover disposed above the sensor platform and covering the sensor electrode; and an adhesive layer formed on the side surfaces of the sensor platform and the cover and bonding the sensor platform and the cover.

To achieve above described objective, a micro sensor package of the present invention is characterized in that and comprises: a sensor platform; a sensor electrode disposed above the sensor platform; a cover disposed above the sensor platform and covering the sensor electrode; and an adhesive layer formed on the outer sides of the sensor platform and the cover and bonding the sensor platform and the cover.

To achieve above described objective, a manufacturing method for a micro sensor package of the present invention is characterized in that and comprises the steps of: stacking of a cover formed with a plurality of cavities on the upper side of a sensor platform formed with a plurality of sensor electrodes; etching for forming a hollow space in the cover and the sensor platform; and injecting an adhesive into the hollow space, wherein at least a portion of the sensor electrode is disposed in the cavities, and the hollow space is disposed between the two sensor electrodes.

In the step of stacking, the sensor platform may be stacked on the upper portion of the substrate.

In the sensor platform, a plurality of first pores is formed along the up-down direction, and the first pores can be formed by anodizing a base material made of a metal.

In the cover, a plurality of second pores is penetratingly formed along the up-down direction, and the second pores can be formed by anodizing a base material made of a metal.

The cavities are penetratingly formed along the up-down direction, and a filter that covers the cavities may be stacked on the upper portion of the cover.

In the filter, a plurality of fourth pores is penetratingly formed along the up-down direction, and the fourth pores can be formed by anodizing a base material made of a metal.

In the step of injecting an adhesive, the adhesive may be intentionally overflowed.

And a step of cutting may further be included for cutting the adhesive along the up-down direction after the step of injecting an adhesive.

In the sensor platform, a heater electrode is formed, and the heater electrode comprises a heating wire disposed closer to a sensor wiring than a sensor electrode pad of the sensor electrode, and an air gap for surrounding the heating wire is formed in the sensor platform, and in the step of stacking, a porous layer formed with a plurality of third pores along the up-down direction may be stacked.

In the sensor platform, a heater electrode is formed, and the heater electrode comprises a heating wire disposed closer to a sensor wiring than a sensor electrode pad of the sensor electrode, and an air gap for surrounding the heating wire is formed in the sensor platform, and the upper side of the air gap is open, and a plurality of first pores may be disposed below the air gap.

### BRIEF DESCRIPTION OF DRAWINGS

FIG. 1 is a view of a vertical cross-section of a miniature package for a gas sensor.

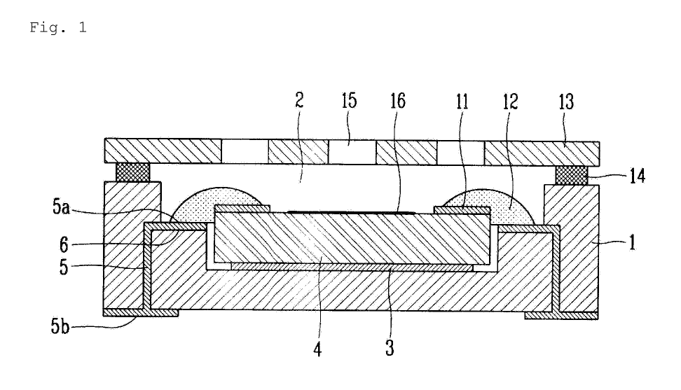

FIG. 2 is a cross-sectional view of a micro sensor package according to the first exemplary embodiment of the present invention.

FIG. 3 is a plan view of a sensing chip according to the first exemplary embodiment of the present invention.

FIG. 4 is an enlarged view of portion ‘A’ in FIG. 3.

FIG. 5 is a cross-sectional view along the line B-B in FIG. 3.

FIG. 6 is a series of cross-sectional views illustrating a manufacturing method of a micro sensor package according to the first exemplary embodiment of the present invention.

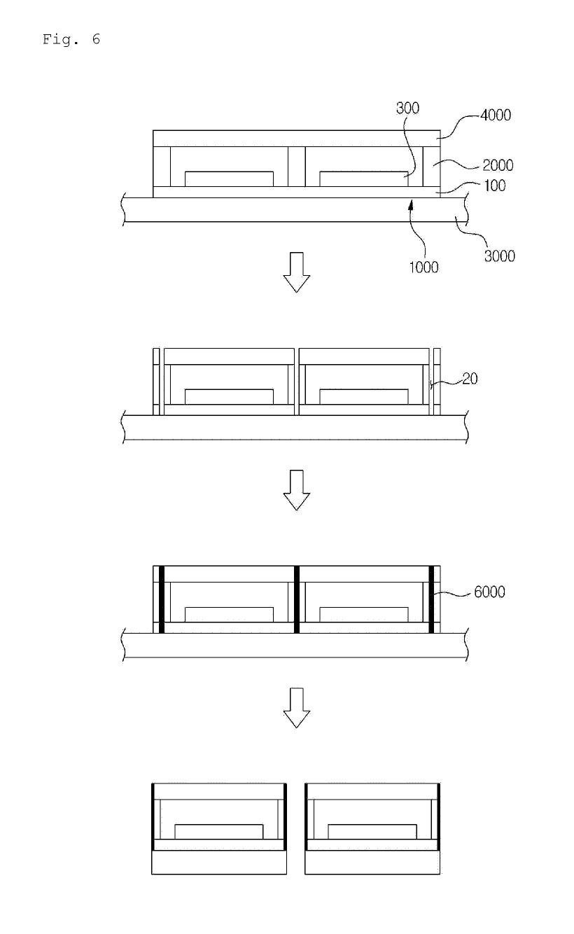

FIG. 7 is a plan view of the etching step when manufacturing a micro sensor package according to the first exemplary embodiment of the present invention.

FIG. 8 is a cross-sectional view of a micro sensor package according to the second exemplary embodiment of the present invention.

FIG. 9 is a cross-sectional view of a micro sensor package according to the third exemplary embodiment of the present invention.

FIG. 10 is a cross-sectional view of a micro sensor package according to the fourth exemplary embodiment of the present invention.

FIG. 11 is a cross-sectional view of a micro sensor package according to the fifth exemplary embodiment of the present invention.

FIG. 12 is a cross-sectional view of a micro sensor package according to the sixth exemplary embodiment of the present invention.

### DETAILED DESCRIPTION OF EMBODIMENTS

Hereinafter, a preferred exemplary embodiment of the present invention will be described in detail with reference to the accompanying drawings as follows.

For reference, for the components of the present invention which will be described hereinafter and identical to those of the prior art, separate detailed descriptions will be omitted, but instead will be referred the prior art described above.

When it is mentioned that one part is on the “top” of other part, this means that the part may be directly on the top of the other part or another different part may be associated with therebetween. In contrast, if it is mentioned that one part is “directly on the top” of the other part, no other part is interposed therebetween.

The terminology used is merely to refer to a particular embodiment and are not intended to limit the present invention. As used herein, the singular forms also include the plural form of text that does not indicate clearly the significance of the opposite. The meaning of “comprising” as used in the specification embodies a specific characteristic, region, integers, steps, operations, elements and/or components, however, it does not exclude the presence or addition of other specific characteristics, regions, integers, steps, operations, elements, components and/or groups.

“Lower,” “upper,” and the like are the terms representing a relative space, and they may be used to describe the relationship of one part with respect to the other part illustrated in the drawing easier. These terms are intended to include other meanings or operations of the device that is used with the meaning intended in the drawing. For example, if the device in the drawing is flipped, the part which was in the “lower” side of the other part is now in the “upper” side of the other part. Thus, the exemplary term “lower” includes all of the upper and lower directions. Device may be rotated 90°, or may be rotated at a different angle, and also the terms indicating the relative space are interpreted accordingly.

### Embodiment 1

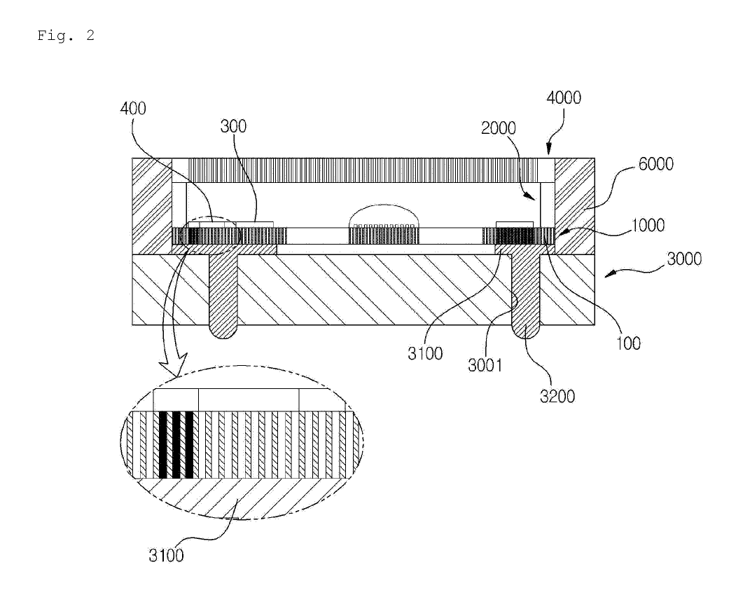

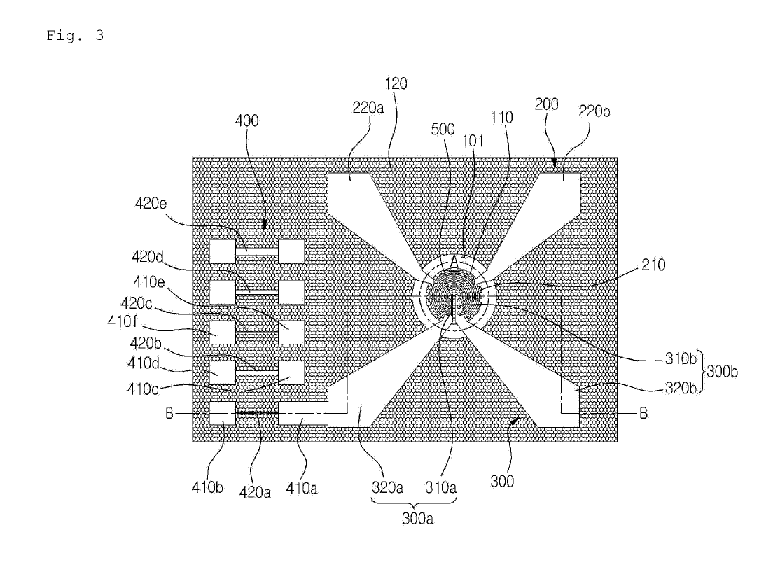

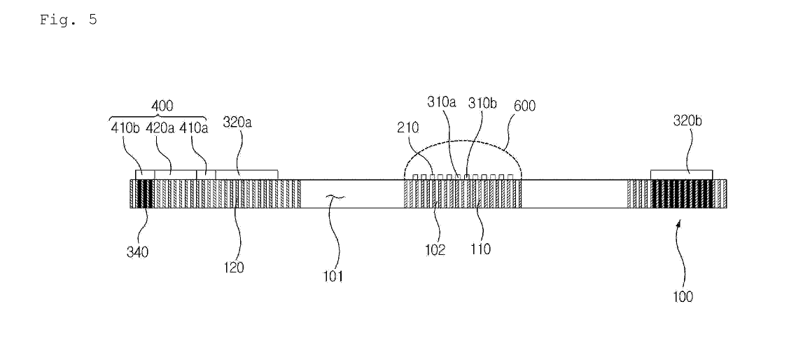

As illustrated in FIGS. 2 to 5, a micro sensor package of an exemplary embodiment is characterized in that and comprises: a sensor platform 100; a sensing chip 1000 comprising a sensor electrode 300 formed in the sensor platform 100; a cover 2000 covering the sensor electrode 300; and an adhesive layer 6000 formed on the side surfaces of the sensor platform 100 and the cover 2000, for bonding the sensor platform 100 and the cover 2000.

The sensing chip 1000 is disposed at the upper portion of the substrate 3000.

The substrate 3000 whose upper and lower surfaces are formed in the shape of a flat plate is disposed horizontally.

The substrate 3000 is formed of an insulating material. Further, the substrate 3000 may be formed of a material having a low thermal conductivity.

A metal pattern 3100 is formed on both sides of the upper surface of the substrate 3000 so as to be spaced apart from each other. The metal pattern 3100 is horizontally formed along the left-to-right direction. A plurality of metal patterns 3100 are formed on the upper surface of the substrate 3000. On the metal pattern 3100, the sensing chip 1000 is mounted. Thus, the substrate 3000 is electrically connected to the sensing chip 1000.

The substrate 3000 is made of PCB or ceramic material. The substrate 3000 is made of a material different from that of the sensor platform 100 and the cover 2000 disposed on the upper portion of the substrate 3000.

A through-hole 3001 is penetratingly formed in the substrate 3000 along the up-down direction at a position corresponding to the metal pattern 3100. A metal portion 3200 is disposed inside the through-hole 3001. The metal portion 3200 is filled in the through-hole 3001.

The upper portion of the metal portion 3200 is connected to the metal pattern 3100.

The metal portion 3200 is formed so as to be protruded below the lowermost end of the substrate 3000.

The micro sensor package is mounted on the PCB through the metal portion 3200.

The sensing chip 1000 is disposed above the substrate 3000 and mounted on the substrate 3000.

The sensing chip 1000 comprises: a sensor platform 100; and a sensor electrode 300 formed on an upper portion or a lower portion of the sensor platform 100 and electrically connected to the metal pattern 3100.

The sensor platform 100 is formed of a porous material formed with a plurality of first pores 102 formed along the up-down direction, thereby improving the heat insulating property.

The left-to-right and front-to-rear widths of the sensor platform 100 are formed to be respectively shorter than the left-to-right and front-to-rear widths of the substrate 3000.

Therefore, the outer side surface of the substrate 3000 is outwardly protruded further from the outer side surface of the sensor platform 100. The side surface means a surface excluding the upper surface and the lower surface.

When an anodizing process is performed on a base material made of a metal, an anodized porous layer having a plurality of pores whose upper side is open is formed. The pores are formed in nanometer size. In here, the base material may be aluminum (Al), titanium (Ti), tungsten (W), zinc (Zn), or the like, but preferably it is made of aluminum or an aluminum alloy material which is lightweight, easy to process, excellent in thermal conductivity, and free from heavy metal contamination.

Further, when the barrier layer and the base material existing under the anodized porous layer are removed, the pores formed in the anodized porous layer are vertically penetrated.

The first pores 102 formed in the sensor platform 100 are formed by anodizing aluminum. Thus, the sensor platform 100 includes an anodized porous layer.

In addition, in the sensor platform 100, aluminum and the barrier layer are removed from the anodized aluminum oxide (AAO), and thereby the first pores 102 are penetrating along the up-down direction.

Unlike to the previous description, the sensor platform may be an anodized film obtained by removing only the base material after anodizing a base material made of a metal. That is, the sensor platform may be an anodized (oxide) film comprising an anodized porous layer and a barrier layer beneath the anodized porous layer.

The sensor platform 100 may be formed of a plate having a rectangular plan shape.

The sensor platform 100 comprises a first support 110 formed at the center of the sensor platform 100, a second support 120 spaced apart from the first support 110, and a bridge portion connecting the first support 110 and the second support 120.

The first support 110 is generally cylindrical in shape, and a plurality of the bridge portions is connected to the outer periphery thereof.

In the sensor platform 100, a plurality of air gaps 101 is formed in the vicinity of the first support 110, that is, between the first support 110 and the second support 120.

The air gap 101 is penetratingly formed along the up-down direction. That is, the air gap 101 is a space formed by penetrating through the sensor platform 100 from the upper surface to the lower surface.

The maximum width (left-to-right width) of the air gap 101 is formed to be wider than the maximum width of the first pore 102 and a sensor wiring or a heating wire 210 which will be described later. The air gap 101 is formed in the shape of an arc, and four of them are formed. A plurality of air gaps 101 is disposed spaced apart along the circumferential direction.

A plurality of air gaps 101 may be discontinuously formed. The air gap 101 and the bridge portion are alternately disposed around the periphery of the first support 110. Therefore, the first support 110 and the second support 120 are spaced apart from each other due to the air gap 101 at portions other than the bridge portion. The bridge portion is formed by discontinuously forming the air gap 101 by etching the vicinity of the first support 110. Thus, one end of the plurality of bridge portions is connected to the first support 110 and the other end is connected to the second support 120. The first support 110 and the second support 120 are connected to each other at four points by the four bridge portions.

The sensor electrode 300 is formed on the upper surface of the sensor platform 100. Thus, the sensor electrode 300 is disposed above the sensor platform 100.

The sensor electrode 300 detects a change in electrical characteristics when the gas is adsorbed to a sensing material 600.

The sensor electrode 300 comprises a first sensor electrode 300a and a second sensor electrode 300b disposed spaced apart from the first sensor electrode 300a. The first sensor electrode 300a and the second sensor electrode 300b are disposed spaced apart from each other and are formed symmetrically with respect to a center line disposed vertically on the plan surface.

Each of the first and second sensor electrodes 300a and 300b comprises the sensor wiring formed on the first supporting portion 110 and a sensor electrode pad formed on the bridge portion and the second support 120.

The first sensor electrode 300a comprises a first sensor wiring 310a formed on the upper surface of the first support 110 and a first sensor electrode pad 320a connected to the first sensor wiring 310a.

The second sensor electrode 300b comprises a second sensor wiring 310b formed on the upper surface of the first support 110 and a second sensor electrode pad 320b connected to the second sensor wiring 310b.

The sensor wiring comprises the first sensor wiring 310a and the second sensor wiring 310b. The sensor electrode pad comprises the first sensor electrode pad 320a and the second sensor electrode pad 320b. The width of the sensor wiring is formed to be constant. The sensor electrode pad is located on the upper surface of the bridge portion and the second support 120 and is formed to have a larger width than the first sensor wiring 310a and the second sensor wiring 310b. The sensor electrode pads of the first and second sensor electrodes 300a and 300b are formed to have a wider width as they travel towards the end portions thereof. That is, the sensor electrode pad is formed to have a narrower width as they travel towards the first sensor wiring 310a and the second sensor wiring 310b.

The sensor electrode 300 is formed of a mixture containing either one or at least one of Pt, W, Co, Ni, Au, and Cu.

A heater electrode 200 is formed on the upper surface of the sensor platform 100.

The upper sides of the first pores 102 located beneath the heater electrode 200 and the sensor electrode 300 are blocked by the heater electrode 200 and the sensor electrode 300 and the lower sides thereof are opened.

The heater electrode 200 comprises: a heating wire 210 formed on the first support 110 so as to be closer to the sensor wire than the sensor electrode pad; and the heater electrode pad connected to the heating wire 210 and formed on the second support 120 and the bridge portion

The heating wire 210 is formed on the upper surface of the first support 110 and is formed by surrounding at least a part of the first sensor wiring 310a and the second sensor wiring 310b from the outside thereof. The heater electrode pad comprises a first heater electrode pad 220a and a second heater electrode pad 220b which are connected to both ends of the heating wire 210 and are spaced apart from each other.

The heating wire 210 is formed to be symmetrical with respect to the vertical center line of the first support 110 and comprises a plurality of arc portions formed in the shape of an arc and a plurality of connecting portions connecting the arc portions.

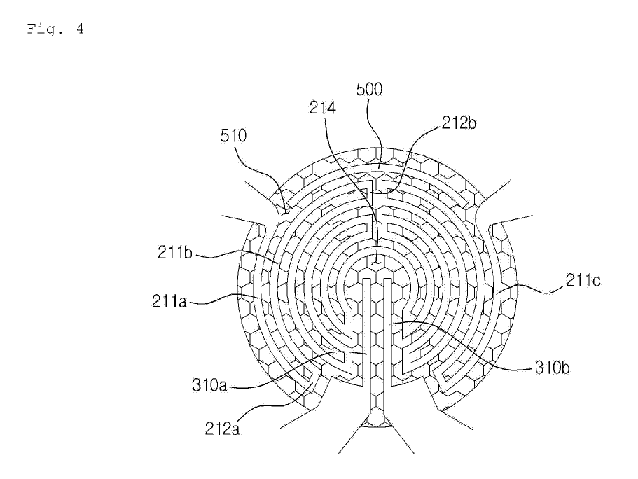

As illustrated in FIG. 4, the heating wire 210 is formed by repeatedly connecting a plurality of arc portions and connecting portions comprising: a first arc portion 211a adjacent to the air gap 101 and formed in the shape of an arc; a first bended portion 212a extending from the one end of the first arc portion 211a toward the inner side of the first support 110; a second arc portion 211b extending in the shape of an arc at an end of the first bended portion 212a and spaced apart from the first arc portion 211a; a second bended portion 212b extending from the end of the second arc portion 211b toward the inner side of the first supporting portion 110; a third arc portion 211c . . . , and so on.

The heating wire 210 is connected from the first arc portion 211a through the third arc portion 211c and forms an integral body.

Each of the plurality of arc portions of the heating wire 210 is formed in the shape of a substantially semicircle so as to form a circular shape as a whole. This improves the temperature uniformity of the first support 110 and the sensing material 600.

The center portion of the heating wire 210 is a point where both arc portions meet with each other, and the two arc portions in the shape of an arc join together to form a circular shape whose one side is open. And a separating space 214 is formed at the inner side thereof. The separating space 214 extends from the central portion of the first support 110 and the heating wire 210 up to the outermost sides of the first supporting portion 110 and the heating wire 210. The sensor wiring is disposed in the separating space 214. In addition, a first heater electrode pad 220a is connected to the other end of the first arc portion 211a and a second heater electrode pad 220b is connected to one end of the third arc portion 211c.

The heater electrode 200 is formed of a mixture containing either one or at least one of Pt, W, Co, Ni, Au, and Cu.

Meanwhile, between the end portions of the first arc portion 211a and the third arc portion 211c to which both ends of the heating wire 210, that is, the first heater electrode pad 220a and the second heater electrode pad 220b are connected, a dummy metal 500 is formed. The dummy metal 500 is formed on the upper surface of the first support 110.

The dummy metal 500 is disposed in the shape of an arc between the heating wire 210 of the heater electrode 200 and the air gap 101. The dummy metal 500 is formed spaced apart from the adjacent heating wire 210.

The dummy metal 500 is formed on the outer side of the heating wire 210 and is preferably a metal. The material of the dummy metal 500 may be the same as that of the electrode material, and the electrode material herein may be a metal such as platinum, aluminum, or copper.

The first arc portion 211a and the third arc portion 211c are formed to have a small central angle as compared with the remaining arc portions at the inner side thereof. A space 510 is formed between the end portions of the first arc portion 211a and the third arc portion 211c in the outer periphery of the heating wire 210, and the dummy metal 500 is located in the space 510.

The space 510 on the outer periphery of the heating wire 210 is partially filled as much as the formation area of the dummy metal 500. Therefore, when viewed in plan, since the outer periphery of the heating wire 210 and the dummy metal 500 form a substantially circular shape, the temperature uniformity of the first support 110 is improved, the temperature distribution of the heating wire 210 on the first support 110 heated with low power becomes more uniform.

The heater electrode pads comprise a first and second heater electrode pads 220a and 220b connected to both ends of the heating wire 210, respectively. In this way, the heater electrode pads are formed in at least two or more. The heater electrode pad is formed so as to have a wider width as it travels towards the outer side. That is, the heater electrode pad is formed to have a narrower width as it travels towards the heating wire 210. The heater electrode pad is formed to have a wider width than the heating wire 210.

The heater electrode pad and the sensor electrode pad are disposed radially with respect to the first support 110. The heater electrode pads and the sensor electrode pads are spaced apart from each other.

A protective layer (not shown) for preventing discoloration is formed on a portion of the upper side of the heater electrode 200 and the sensor electrode 300. The protective layer for preventing discoloration may be formed of an oxide-based material. Further, the protective layer for preventing discoloration is formed of at least one of tantalum oxide (TaOx), titanium oxide (TiO₂), silicon oxide (SiO₂), and aluminum oxide (Al₂O₃).

The heating wire 210 and the first and second sensor wirings 310a and 310b are surrounded by the air gap 101. The air gap 101 is disposed around the heating wire 210 and the first and second sensor wirings 310a and 310b. The air gap 101 is disposed at the side of the heating wire 210 and the first and second sensor wirings 310a and 310b.

More specifically, the air gap 101 is formed between the first sensor electrode pad 320a and the first heater electrode pad 220a of the first sensor electrode 300a and between the first heater electrode pad 220a and the second heater electrode pad 220b and between the second heater electrode pad 220b and the second sensor electrode pad 320b of the second sensor electrode 300b and between the second sensor electrode pad 320b of the second sensor electrode 300b and the first sensor electrode pad 320a of the first sensor electrode 300a. That is, the air gap 101 is formed in a region excluding the portion supporting the heater electrode 200 and the sensor electrode 300.

Due to the air gap 101, the first support 110 which commonly supports the heating wire 210 and the sensor wiring, the second support 120 which supports the heater electrode pad and the sensor electrode pad, and the bridge portion are formed in the sensor platform 100.

The first support 110 is formed to have a larger area than the heating wire 210 and the sensor wiring.

The first support 110 is formed with a heating wire 210 and a sensing material 600 covering the sensor wiring. That is, the sensing material 600 is formed at a position corresponding to the first support 110. The sensing material 600 is formed by printing. In this way, once the sensing material 600 is formed by printing, a trace resembling a mesh network is left on the surface of the sensing material 600 after the sensing material 600 is formed.

Further, a resistor array 400 electrically connected to the sensor electrode pad of the sensor electrode 300 is formed on the sensor platform 100.

The resistor array 400 is formed on the upper surface of the sensor platform 100 and formed on the same plane as the sensor electrode 300.

The resistor array 400 is disposed spaced apart from the heater electrode 200.

The resistor array 400 is disposed at the second support 120.

In the present exemplary embodiment, the gas sensing portion (sensor electrode and heater electrode) is disposed at the right side of the sensor platform 100, and the resistor array 400 is disposed at the left side of the sensor platform 100. Accordingly, the air gap 101 is disposed between the resistor array 400 and the first support 110.

The resistor array 400 includes at least two resistors.

At least one of the resistors is formed in the shape of a sheet resistor or a fine pattern (line shape), so that the volume of the resistor array 400 can be minimized.

More specifically, the resistor array 400 comprises first, second, third, fourth and fifth resistor pads 410a, 410b, 410c, 410d and 410e and first, second and third resistors 420a, 420b and 420c.

Each of the resistor pads is disposed to be spaced apart from each other.

The first resistor pad 410a is connected to the first sensor electrode pad 320a of the sensor electrode 300. Unlike to the previous description, the first resistor pad may be connected to the second sensor electrode pad of the sensor electrode 300.

The first resistor pad 410a of the resistor array 400 connected to the sensor electrode 300 is integrally formed with the first sensor electrode pad 320a or the second sensor electrode pad 320b of the sensor electrode 300. Therefore, the resistor array 400 is formed integrally with the sensor electrode 300. Unlike to this, the resistor array and the sensor electrode are formed separately, but the resistor array may be installed or formed on the sensor platform.

The first resistor pad 410a is connected to at least one other resistor pad via at least one resistor.

The first resistor pad 410a is connected to one side of the first resistor 420a and the second resistor pad 410b is connected to the other side of the first resistor 420a.

The third resistor pad 410c is connected to one side of the second resistor 420b and the fourth resistor pad 410d is connected to the other side of the second resistor 420b.

A fifth resistor pad 410e is connected to one side of the third resistor 420c and the sixth resistor pad 410f is connected to the other side of the third resistor 420c.

In the present exemplary embodiment, the resistor array may comprise five resistors. The resistor array comprises first, second, third, fourth and fifth resistors 420a, 420b, 420c, 420d and 420e.

Each of the resistors in the present exemplary embodiment has resistive pads on both sides. Therefore, the resistance pads are connected to one side and the other side of the fourth and fifth resistors 420d and 420e, which are the remaining resistors.

Each of the five resistors may have a different resistance value, or at least two of the five resistors have different resistance values.

The first resistor 420a connected to the first sensor electrode pad 320a of the sensor electrode 300 may have the largest value among the five resistors. The first, second, third, fourth and fifth resistors 420a, 420b, 420c, 420d and 420e are provided as sheet resistors, and the resistance becomes larger as the line width (front-to-rear width) becomes thinner.

The resistor array 400 is connected only to the first sensor electrode pad 320a of the sensor electrode 300 and the sensor electrode 300 is connected to the resistor array 400 in series.

Each of the resistor pads may be selectively connected to the sensor electrode 300 through wire bonding or the like depending on the resistance of the sensing material 600.

At least two of the resistors may be connected in series or in parallel.

The first pores 102 beneath the sensor electrode pads or the resistor pads or the heater electrode pads are penetratingly formed along the up-down direction. The first pores 102 disposed between the sensor electrode pad or the resistor pad or the heater electrode pad and the metal pattern 3100 is penetratingly formed along the up-down direction.

That is, the first pores 102 are formed so as to penetrate from the surface, wherein the sensor electrode pads or the resistor pads or the heater electrode pads are formed, into the opposite surface.

Inside the plurality of first pores 102 beneath the sensor electrode pad or the resistance pad or the heater electrode pad, a first connecting portion 340 is formed for electrically connecting a heater electrode pad of the sensor electrode 300 or a heater electrode pad of the heater electrode 200 to the metal pattern 3100 disposed at the opposite side of the sensor electrode 300 and the heater electrode 200. That is, the first connecting portion 340 is filled in the first pores 102. The lower portion of the first connecting portion 340 can be formed so as to be protruded lower than the lower surface of the sensor platform 100. Due to this, the connection between the first connecting portion 340 and the metal pattern 3100 may become smoother.

The connection means a direct connection or an indirect connection. Unlike to the above description, in the sensor platform, a sensor bonding portion connected to a lower portion of the first connecting portion may be further formed on a bottom surface which is opposite to the surface on which the sensor electrode, the heater electrode, and the resistor are formed. The upper portion of the sensor bonding portion is horizontally formed along the left-to-right direction so as to be connected to the plurality of first connecting portions. A metal pattern may be connected to a lower portion of the sensor bonding portion. That is, the first connecting portions and the metal pattern may be indirectly connected through the sensor bonding portion.

The first connecting portion 340 is formed in the shape of a column having a diameter of several nanometers. The connecting portion disposed inside the pore may be formed of a conductive metal material.

In the present exemplary embodiment, since the resistor array 400 is integrally formed on the first sensor electrode pad 320a of the sensor electrode 300, at least one of the five resistor pads 410b, 410c, 410d, and 410e or the second sensor electrode pad 320b, and the first and second heater electrode pads 220a and 220b are connected to the upper portion of the first connecting portion 340.

In this way, the first connecting portion 340 is formed inside the first pores 102 so that the first connecting portion 340 can be formed without any additional etching operation and may be mounted in the form of a surface mount device (SMD) without wire bonding.

The cover 2000 covers the sensor electrode 300, the heater electrode 200, and the resistor array 400 formed on the upper surface of the sensing chip 1000.

The cover 2000 is disposed at the upper portion of the sensor platform 100 of the sensing chip 1000.

The left-to-right and front-to-rear widths of the cover 2000 are formed equal or similar to the left-to-right and front-to-rear widths of the sensor platform 100. That is, the outer shape of the cover 2000 is formed to correspond to the outer shape of the sensor platform 100. Therefore, the outer side surface of the cover 2000 is formed continuously on the outer side surface of the sensor platform 100.

In the cover 2000, a cavity in which at least a portion of the sensor electrode 300 is disposed is formed.

The cavity is penetratingly formed along the up-down direction. That is, the cavity is formed so that the upper and lower portions are open.

The cover 2000 surrounds the sensor electrode 300, the heater electrode 200, and the side portion of the resistor array 400.

The cover 2000 is formed in the shape of a porous plate wherein the second pores are formed along the up-down direction. Further, the second pores are formed by anodizing aluminum. In such a cover 2000, the cavity is formed through etching or the like. In this way, the cover 2000 attached to the sensor platform 100 is formed of the same material as the sensor platform 100 so that it can be etched at one time during manufacturing.

A filter 4000 is provided so as to cover the cavity.

Therefore, the gas is supplied to the sensing chip 1000 after passing through the filter 4000.

The filter 4000 is formed in the shape of a plate and disposed in the upper side of the cover 2000. Thus, the filter 4000 is installed in the outer side of the cover 2000.

The filter 4000 may be formed of a porous material.

Further, the filter 4000 may be formed of an anodized aluminum porous layer wherein a plurality of fourth pores is penetratingly formed along the up-down direction through anodizing process. The fourth pores communicate with the cavity.

The inside of the fourth pore of the filter 4000 is subjected to a hydrophobic surface treatment to prevent moisture from infiltrating into the gas detecting portion.

The filter 4000 may be surface treated such that a specific gas is selectively passing through or being blocked. Unlike to this, the filter 4000 may have a different diameter of the fourth pore for selective passing of gas. That is, the diameter of the fourth pore may be different depending on the type of gas to be detected.

The adhesive layer 6000 is formed on the outer side surfaces of the sensor platform 100 and the cover 2000 and the filter 4000 so as to bond the sensor platform 100 and the cover 2000 to the filter 4000.

That is, the adhesive layer 6000 is formed on the outer side of the sensor platform 100, the cover 2000, and the filter 4000. The adhesive layer 6000 is penetratingly formed along the up-down direction so as to intersect between the sensor platform 100 and the cover 2000 and between the cover 2000 and the filter 4000.

That is, the adhesive layer 6000 connectively bonds the outer side surfaces of the sensor platform 100, the cover 2000, and the filter 4000 to each other.

The adhesive layer 6000 surrounds the outer side surfaces of the sensor platform 100 and the cover 2000 and the filter 4000 so as to prevent foreign substances or unfiltered gas from being introduced through between the sensor platform 100 and the cover 2000 or between the cover 2000 and the filter 4000.

The adhesive layer 6000 surrounds at least a portion of the side surfaces or the entire circumference of the sensor platform 100, the cover 2000, and the filter 4000.

The adhesive layer 6000 is formed so as to surround the outer side surface of the metal pattern 3100 as well.

In addition, the adhesive layer 6000 is also formed on the upper surface of the portion protruding outward from the substrate 3000 to connect the outer side surface of the sensor platform 100 and the upper surface of the substrate 3000, thereby bonding the sensor platform 100 with the substrate 3000.

The adhesive layer 6000 for connecting each of the members is integrally formed.

The outer side surface of the adhesive layer 6000 is formed continuously on the outer surface of the substrate 3000.

Hereinafter, the operation of the present exemplary embodiments having the above described configuration will be described.

In order to measure the gas concentration, a constant electric power is first applied to the two heater electrode pads of the heater electrode 200 to heat the sensing material 600 to a constant temperature.

In the heated sensing material 600, the gas inside the cavity that has been passed through the filter 4000 is adsorbed or desorbed.

As a result, the electrical conductivity between the first sensor wiring 310a and the second sensor wiring 310b changes, and the sensing signal is amplified through the resistor array 400 to detect the gas.

Further, in order to perform more precise measurement, other gas species or moisture already adsorbed to the sensing material 600 are heated at a high temperature by the heater electrode 200 so as to be forcibly removed from the sensing material 600, and thereby, the sensing material 600 is recovered to its initial state so that the gas concentration is measured.

A manufacturing method for a micro sensor package of the present exemplary embodiment is characterized in that and comprises the steps of: stacking of a cover 2000 formed with a plurality of cavities on the upper side of a sensor platform 100 formed with a plurality of sensor electrodes 300 and heater electrodes 200; etching for forming a hollow space 20 in the cover 2000 and the sensor platform 100; and injecting an adhesive into the hollow space 20, wherein at least a portion of the sensor electrode 300 is disposed in the cavities, and the hollow space 20 is disposed between the two sensor electrodes 300.

In the step of stacking, a cover 2000 formed with a plurality of cavities is stacked on a sensor platform 100 on which a plurality of sensor electrodes 300 and heater electrodes 200, and a resistor array 400 are formed.

The cover 2000 is stacked so that at least a portion of the sensor electrodes 300 and the heater electrodes 200 are disposed in the cavities.

The sensor platform 100 is stacked on the upper side of a substrate 3000 on which a metal pattern 3100 is formed. When it is stacked, the sensor electrodes 300, the heater electrodes 200, and the resistor array 400 are electrically connected to a metal pattern 3100.

The substrate 3000 is provided with a PCB.

In addition, the filter 4000 that covers the cavities is stacked on the upper side of the cover 2000.

That is, a sensing chip 1000 in the shape of a flat plate is stacked on the substrate 3000 in the shape of a flat plate, and the cover 2000 in the shape of a flat plate formed to penetrate the cavity along the up-down direction is stacked on the sensing chip 1000, and the filter 4000 in the shape of a flat plate is stacked on the cover 2000. Due to this, the sensor electrodes 300, the heater electrodes 200, and the resistor array 400 are disposed inside the cavity, which is vertically and horizontally closed.

The sensor platform 100, the cover 2000, and the filter 4000 are formed of an AAO material wherein a plurality of pores is formed along the up-down direction by anodizing aluminum (metal material).

The fourth pores of the filter 4000 are penetratingly formed along the up-down direction.

Subsequently, a plurality of hollowed spaces 20 are formed through etching on the sensor platform 100 and the cover 2000 and the filter 4000 stacked on the substrate 3000. The hollowed spaces 20 are formed along the up-down direction. The sensor platform 100, the cover 2000, and the filter 4000 are formed of AAO material and are etched along the up-down direction during etching. In the step of etching, the substrate 3000 is not etched.

The hollow spaces 20 are disposed between the two (adjacent) sensor electrodes 300.

The hollow spaces 20 are disposed between the two cavities.

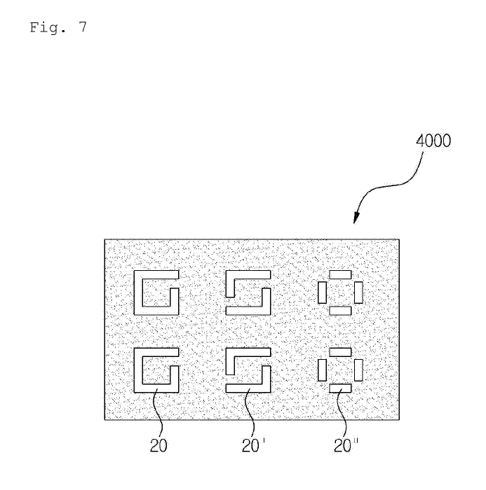

The hollowed spaces 20 are formed to surround the sides of the cavities as illustrated in FIG. 7.

The hollow spaces 20, 20′ and 20″ are formed in the shape of a letter ‘G’ or ┌┘ or a square with its edges separated so that the inner sides and outer sides of the hollow spaces 20 are connected at one or two or four places.

That is, the hollow spaces 20, 20′ and 20″ are formed so that the inner side and outer side of the hollow space 20 are connected at least one place.

Unlike to this, as in the present exemplary embodiment, when the substrate 3000 of a material different from that of the sensor platform 100 is provided, a hollow space 20 may be formed to surround the entire circumference of the cavity.

In this way, after the hollow space 20 is formed, an adhesive layer 6000 is formed by injecting an adhesive in to the hollow space 20.

Thus, the adhesive layer 6000 is formed along the up-down direction between the unit micro sensor packages.

After the step of injecting, the adhesive layer 6000 and the substrate 3000 are cut along the adhesive along the up-down direction. Due to this, the unit micro sensor packages are formed.

In this way, since dicing is performed along the line of the adhesive, the dicing area of the brittle sensor platform 100, the cover 2000, and filter 4000 is minimized or eliminated, so that breaking of the sensor platform 100, the cover 2000, and filter 4000 is prevented during the dicing process.

In addition, even after the dicing, such an adhesive layer 6000 plays the role of connectively combining the upper surface of the substrate 3000 and the outer side surfaces of the sensor platform 100, the cover 2000, and filter 4000.

### Embodiment 2

In describing the micro sensor package according to the second exemplary embodiment of the present invention, same symbols will be used for the same or similar elements as those of the micro sensor package according to the first exemplary embodiment of the present invention, and the detailed description and illustration will be omitted.

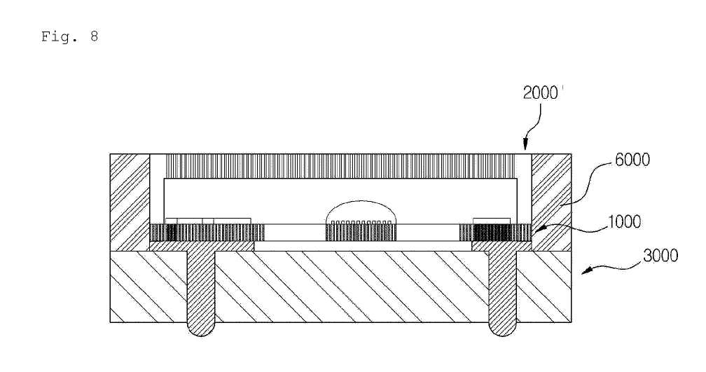

As illustrated in FIG. 8, in a micro sensor package according to the second exemplary embodiment, a plurality of second pores for supplying a gas to a sensing chip 1000 is penetratingly formed in a cover 2000′ along the up-down direction.

The second pores can be formed by anodizing a base material made of a metal material.

Preferably, the second pores are formed with AAO.

Such cover 2000′ also plays the role of a filter simultaneously. That is, the cover 2000′ of the present exemplary embodiment is integrally formed with the filter.

A cavity formed in the cover 2000′ of the present exemplary embodiment is formed through etching so that only the lower portion of the cavity is open. The cavity is blocked by the porous layer provided with the second pores penetrated along the up-down direction.

The sensing chip 1000 is stacked on the upper side of a substrate 3000 and the cover 2000′ is stacked on the upper side of a sensor platform of the sensing chip 1000.

An adhesive layer 6000 is formed on the upper surface of the substrate 3000 and on the outer side surfaces of the sensor platform and the cover 2000′, thereby bonding the substrate 3000, the sensor platform, and the cover 2000′ to one another.

### Embodiment 3

In describing the micro sensor package according to the third exemplary embodiment of the present invention, same symbols will be used for the same or similar elements as those of the micro sensor package according to the first and second exemplary embodiments of the present invention, and the detailed description and illustration will be omitted.

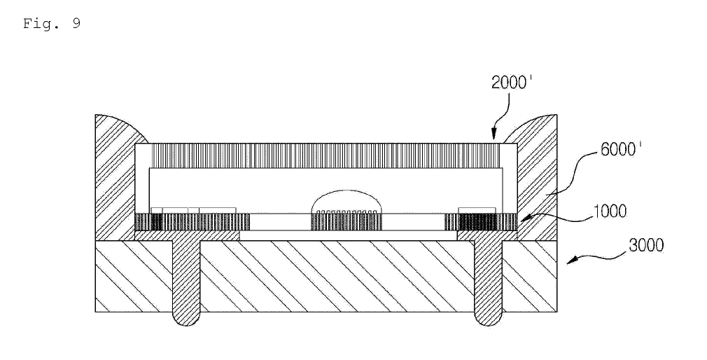

As illustrated in FIG. 9, in a micro sensor package according to the third exemplary embodiment, an adhesive layer 6000′ is formed also on the upper surface of a cover 2000′.

The cover 2000′ of the present exemplary embodiment is formed of an AAO material so as to simultaneously perform the role of a filter as in the second exemplary embodiment, and a cavity is formed so that only the lower side thereof is open.

A sensing chip 1000 is stacked on the upper side of a substrate 3000, and the cover 2000′ is stacked on the upper side of a sensor platform of the sensing chip 1000.

An adhesive layer 6000′ is formed on the upper surface of the substrate 3000 and on the outer side surfaces of the sensor platform and the cover 2000′ and on a portion of the upper surface of the cover 2000′ so that the bonding strength can be enhanced further. The adhesive layer 6000′ is formed along the edge of the upper surface of the cover 2000′.

The upper surface of the adhesive layer 6000′ is formed further upwardly protruded than the upper surface of the cover 2000′ disposed in the uppermost end of the package.

Such adhesive layer 6000′ is formed by overflowing an adhesive in the step of injecting the adhesive into a hollow space when manufacturing the micro sensor package.

### Embodiment 4

In describing the micro sensor package according to the fourth exemplary embodiment of the present invention, same symbols will be used for the same or similar elements as those of the micro sensor package according to the first, second, and third exemplary embodiments of the present invention, and the detailed description and illustration will be omitted.

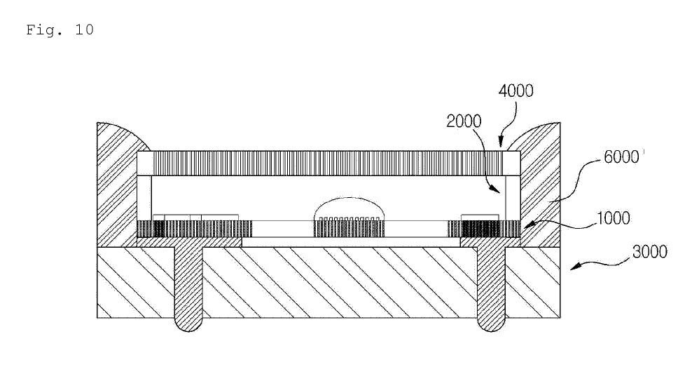

As illustrated in FIG. 10, in a micro sensor package according to the fourth exemplary embodiment, a cover 2000 and a filter 4000 are separately provided, and an adhesive layer 6000′ is formed also on the upper surface of the filter 4000 disposed at the uppermost end of this package.

A sensing chip 1000 is stacked on the upper side of a substrate 3000, and a cover 2000 is stacked on the upper side of a sensor platform of the sensing chip 1000, and the filter 4000 is stacked on the upper side of the cover 2000.

The adhesive layer 6000′ is formed on the upper surface of the substrate 3000, on the outer side surfaces of the sensor platform and the cover 2000 and the filter 4000, and on a portion of the upper surface of the filter 4000.

Thus, the substrate 3000, the sensor platform, the cover 2000, and the filter 4000 are bonded to one another.

### Embodiment 5

In describing the micro sensor package according to the fifth exemplary embodiment of the present invention, same symbols will be used for the same or similar elements as those of the micro sensor package according to the first, second, third, and fourth exemplary embodiments of the present invention, and the detailed description and illustration will be omitted.

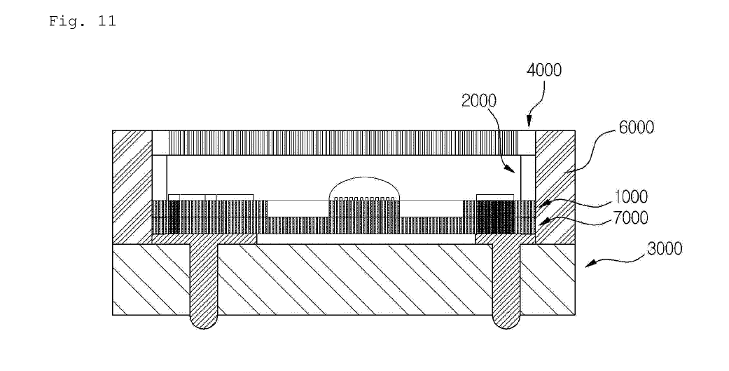

As illustrated in FIG. 11, in a micro sensor package according to the fifth exemplary embodiment, a porous layer 7000 disposed in the lower side of a sensor platform of a sensing chip 1000 and formed with a plurality of third pores along the up-down direction is further included.

The porous layer 7000 is stacked on a substrate 3000, and the sensing chip 1000 is stacked on the porous layer 7000, and the cover 2000 is stacked on the sensor platform, and a filter 4000 is stacked on the upper side of the cover 2000.

The porous layer 7000 is formed by anodizing a base material made of a metal material.

The porous layer 7000 is disposed in the lower side of an air gap formed in the sensor platform so as to block the lower side of the air gap. Due to this, foreign substances are prevented from being introduced into the sensing material of the sensing chip 1000 through the lower portion of the air gap. That is, the porous layer 7000 plays the role of a filter. In addition, owing to this porous layer 7000, the thermal conductivity can be reduced.

At least a portion of a plurality of third pores of the porous layer 7000 is penetratingly formed along the up-down direction, and thereby a second connecting portion is formed inside the third pores penetrating along the up-down direction. The second connecting portion is electrically connected to a first connecting portion of the sensing chip 1000 and a metal pattern of the substrate 3000.

The adhesive layer 6000 is formed on the upper surface of the substrate 3000, on the outer side surfaces of the porous layer 7000, the sensor platform, the cover 2000, and the filter 4000.

Thus, the substrate 3000, the porous layer 7000, the sensor platform, the cover 2000, and the filter 4000 are bonded to one another.

Unlike to the previous description, the sensor platform and the porous layer may be integrally formed instead of providing them separately. In this case, the sensor platform is provided with a plurality of first pores formed along the up-down direction, and the air gap formed in the sensor platform is formed not to penetrate along the up-down direction, but formed in a way that the upper portion thereof is open while the lower portion is closed. Further, it is formed in a way that a plurality of first pores is disposed below the air gap.

### Embodiment 6

In describing the micro sensor package according to the sixth exemplary embodiment of the present invention, same symbols will be used for the same or similar elements as those of the micro sensor package according to the first, second, third, fourth, and fifth exemplary embodiments of the present invention, and the detailed description and illustration will be omitted.

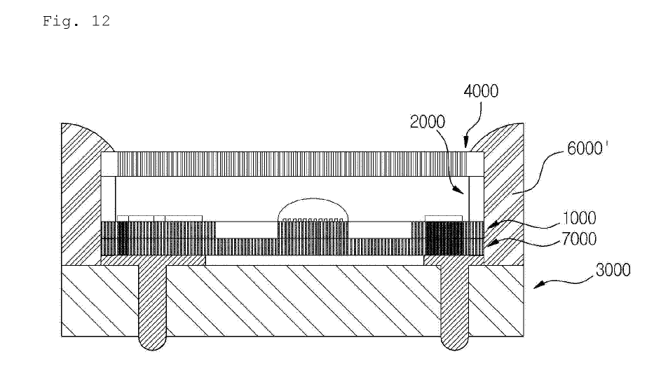

As illustrated in FIG. 12, in a micro sensor package according to the sixth exemplary embodiment, a porous layer 7000 is disposed below a sensor platform of a sensing chip 1000, and an adhesive layer 6000′ is formed also on the upper surface of a filter 4000 disposed at the uppermost end of this package.

The porous layer 7000 is stacked on the upper side of a substrate 3000, and a sensing chip 1000 is stacked on the upper side of the porous layer 7000, and a cover 2000 is stacked on the upper side of a sensor platform, and a filter 4000 is stacked on the upper side of the cover 2000.

The adhesive layer 6000′ is formed on the upper surface of the substrate 3000, on the outer side surfaces of the porous layer 7000, the sensor platform, the cover 2000, and the filter 4000.

Thus, the substrate 3000, the porous layer 7000, the sensor platform, the cover 2000, and the filter 4000 are bonded to one another.

According to the micro sensor package and a manufacturing method thereof according to the present invention as described above, the following effects can be obtained.

By side-to-side surface-bonding of a sensor platform to (with) a cover through an adhesive layer, unfiltered gas is prevented from being supplied to the sensor electrode.

By further comprising a substrate electrically connected to the sensing chip, the durability of the micro sensor package can be enhanced further.

The adhesive layer is also formed on the upper surface of the substrate, and thereby the sensing chip and the substrate are bonded to each other, therefore, the substrate, the sensor platform, and the cover can be bonded together through a single process, which further simplifies the manufacturing process.

In the sensor platform, a plurality of first pores may be formed along the up-down direction so that the thermal conductivity can be reduced. In addition, since it is etched vertically without having the directivity during etching, etching can be performed easily.

A plurality of second pores for supplying gas to the sensing chip is penetratingly formed in the cover along the up-down direction so that the cover can function as a filter at the same time, thereby further facilitating manufacturing.

The adhesive layer is also formed on the side surface of the filter, and thereby the filter and the cover are bonded to each other, therefore, the sensor platform, the cover, and the filter can be bonded together through a single process, which further simplifies the manufacturing process.

In the sensor platform, a heater electrode is formed, and the heater electrode comprises a heating wire disposed closer to a sensor wiring than a sensor electrode pad of the sensor electrode, and an air gap for surrounding the heating wire is formed in the sensor platform, and a porous layer disposed below the sensing chip and is penetratingly formed with a plurality of third pores along the up-down direction may further be included, or the upper side of the air gap is open, and a plurality of first pores is disposed below the air gap, so that foreign substances can be prevented from being introduced through the lower portion of the air gap, and the thermal conductivity can be reduced.

The first pores are penetratingly formed along the up-down direction, and a first connecting portion electrically connected the sensor electrode is formed inside at least a portion of the plurality of first pores, so that the micro sensor package can be formed compactly.

The adhesive may be intentionally overflowed so that the adhesive strength can be enhanced.

By cutting along the up-down direction along the adhesive, the sensor platform, the cover, and the filter are prevented from breaking in the cutting process even when the sensor platform, the cover, or the filter is made of a brittle material such as AAO.

As described above, although the present invention has been described with reference to the preferred exemplary embodiments, various changes and alterations of the present invention can be made by those skilled in the art without departing from the spirit and the scope of the present invention written in the claims described herein below.

1. A micro sensor package comprising:
a sensing chip comprising a sensor platform and a sensor electrode formed in the sensor platform;
a cover covering the sensor electrode; and
an adhesive layer formed on side surfaces of the sensor platform and the cover, for bonding the sensor platform and the cover.

2. The micro sensor package according to claim 1, further comprising:
a substrate electrically connected to the sensing chip.

3. The micro sensor package according to claim 1,
wherein the adhesive layer is also formed on an upper surface of the substrate so that the sensing chip and the substrate are bonded together.

4. The micro sensor package according to claim 1,
wherein in the sensor platform, a plurality of first pores are formed along an up-down direction.

5. The micro sensor package according to claim 1,
wherein in the cover, a plurality of second pores for supplying a gas to the sensing chip are formed along an up-down direction.

6. The micro sensor package according to claim 1, further comprising:
a filter that covers the cavity,
wherein a cavity disposed with at least a portion of the sensor electrode is formed in the cover, and the cavity is penetratingly formed along an up-down direction.

7. The micro sensor package according to claim 6,
wherein a plurality of fourth pores are penetratingly formed in the filter along the up-down direction and the fourth pores communicate with the cavity.

8. The micro sensor package according to claim 6,
wherein the adhesive layer is also formed on a side surface of the filter so that the filter is bonded with the cover.

9. The micro sensor package according to claim 1, further comprising:
a porous layer disposed below the sensing chip and penetratingly formed with a plurality of third pores along an up-down direction,
wherein in the sensor platform, a heater electrode is formed,
wherein the heater electrode comprises a heating wire disposed closer to a sensor wiring than a sensor electrode pad of the sensor electrode, and
wherein an air gap for surrounding the heating wire is formed in the sensor platform.

10. The micro sensor package according to claim 4,
wherein in the sensor platform, a heater electrode is formed,
wherein the heater electrode comprises a heating wire disposed closer to a sensor wiring than a sensor electrode pad of the sensor electrode,
wherein an air gap that surrounds the heating wire is formed in the sensor platform,
wherein an upper side of the air gap is open, and
wherein a plurality of first pores is disposed below the air gap.

11. The micro sensor package according to claim 4,
wherein the first pores are penetratingly formed along the up-down direction, and a first connecting portion which is electrically connected to the sensor electrode is formed inside at least a portion of the plurality of first pores.

12. A micro sensor package comprising:
a sensor platform;
a sensor electrode disposed above the sensor platform;
a cover disposed above the sensor platform and covering the sensor electrode; and
an adhesive layer formed on side surfaces of the sensor platform and the cover to bond the sensor platform and the cover.

13. A micro sensor package comprising:
a sensor platform;
a sensor electrode disposed above the sensor platform;
a cover disposed above the sensor platform and covering the sensor electrode; and
an adhesive layer formed on the outer sides of the sensor platform and the cover to bond the sensor platform and the cover.
